(19) United States
(12) Patent Application Publication
(10) Pub. No.: US 2023/0029995 A1
(43) Pub. Date: Feb. 2, 2023

(54) HEART RATE AND BLOOD OXYGEN MONITORING DEVICE

(71) Applicant: Luxsentek Microelectronics Corp., New Taipei City (TW)

(72) Inventors: SHENG-CHENG LEE, New Taipei City (TW); WEN-SHENG LIN, New Taipei City (TW); CHIH-WEI LIN, New Taipei City (TW); CHEN-HUA HSI, New Taipei City (TW); YUEH-HUNG HOU, New Taipei City (TW)

(21) Appl. No.: 17/483,272

(22) Filed: Sep. 23, 2021

(30) Foreign Application Priority Data
Jul. 26, 2021 (TW) 110127372

(51) Int. Cl. A61B 5/024; A61B 5/00; A61B 5/1455

(52) U.S. Cl. CPC A61B 5/02438; A61B 5/7225; A61B 5/7203; A61B 5/002; A61B 5/6801; A61B 5/14551; A61B 5/0004

(57) ABSTRACT

The heart rate and blood oxygen monitoring device of the present invention utilizes two ambient light calibration DACs, which can quickly calibrate noise signals generated by ambient light. The heart rate and blood oxygen monitoring device can provide more accurate heart rate and blood oxygen values for optical measurement methods.

### BACKGROUND OF THE INVENTION

### 1. Field of the Invention

The present invention relates to a heart rate and blood oxygen monitoring device, and particularly that can avoid or reduce the interference from an ambient light.

### 2. Description of the Prior Art

Smart wearable devices can measure human's physiological signals, that use non-invasive optical sensing device usually, for example, the pulse and blood oxygen measurement. Now that has been used to real-time monitor the health conditions.

Heart contracts and relaxes to transport the blood to cause the pulse, that is a regular variation of the blood vessels and corresponding to the heart rate. Heart contraction pushes oxygen-rich blood into the blood vessels, and the amount of the oxygenated hemoglobin (HbO₂) and the non-oxygenated hemoglobin (Hb) changes in regular accordingly. A light absorption of the HbO₂ is different from the Hb, especially the red-light and the infrared-ray. When the red-light and/or infrared ray irradiates on the skin and some penetrates the skin, a part of the penetration light will be absorbed by the HbO₂ and the Hb and the other part is reflected, so the reflection signal varies with the pulse. The reflection light can be captured by the sensor and used to measure the heart rate and the blood oxygen. FIG. 1. is a photoplethysmography (PPG), that is formed by an irradiation light pulse with a fixed period (mark as the pulse repetition period). The PPG includes an AC component and a DC component, and the blood oxygen saturation can be calculated to compare the amplitude of DC component and AC component.

A heart rate and blood oxygen monitoring device is generally designed to be a finger-clamping device, or a body patch and the heart rate and blood oxygen monitoring device comprises light emitting elements, light sensing elements/photodiode, and a control module. The light emitting element is used to illuminate the light, the light sensing element/photodiode to receive the reflection light of the HbO₂ and Hb and convert into the electrical signal, and the control module to control the components and analysis the measurements.

However, ambient light and/or the reflection of the skin, surrounding tissues of arteries, bones or veins are also absorbed to seriously affect the measurement result. The present invention provides a solution to avoid/reduce the interference caused by ambient light.

### SUMMARY OF THE INVENTION

The present invention provides a solution to reduce ambient light interference quickly and a fine hear rate and SPO₂ measurement.

A heart rate and blood oxygen monitoring device, comprising:
a red-light emitting element driven by a red-light driver to emit a red-light detection light, and an infrared-ray emitting element driven by an infrared-ray driver to emit an infrared-ray detection light;
a light receiving module connected to an analog front-end module through a first switch unit to convert the detection lights to an analog signal;
an analog-to-digital converter connected to the analog front-end module through a second switch unit to convert the analog signal to an operation digital signal;
a one-cycle-clock ADC connected to the analog front-end module through a third switch unit to convert the analog signal to a calibration digital signal;
a digital signal processor connected to the analog-to-digital converter and the one-cycle-clock ADC to convert the calibration digital signal and the operation digital signal to a calibration parameter and a measurement data respectively;
a first ambient light calibration DAC connected to the light receiving module through a fourth switch unit to transmit an ambient light calibration analog signal to the light receiving module;
a second ambient light calibration DAC connected to the light receiving module through a fifth switch unit to transmit an ambient light calibration analog signal to the light receiving module;
a timing controller configured to drive the red-light driver, the infrared-light driver, the first ambient light calibration DAC, and the second ambient light calibration DAC;
a microcontroller connected to the digital signal processor and the timing controller, the microcontroller controls the heart rate and blood oxygen monitoring device into an operation mode or a calibration mode by switching the first switch unit, the second switch unit, the third switch unit, and the fourth switch unit, wherein the red-light detection light and the infrared-ray detection light can be partly absorbed and partly reflected to measure a heart rate and a blood oxygen saturation value.

The heart rate and blood oxygen monitoring device can provide a medical measurement.

The heart rate and blood oxygen monitoring device can be integrated into a wearable device.

### BRIEF DESCRIPTION OF THE DRAWINGS

FIG. 1 shows the photoplethysmography of heart rate and blood oxygen.

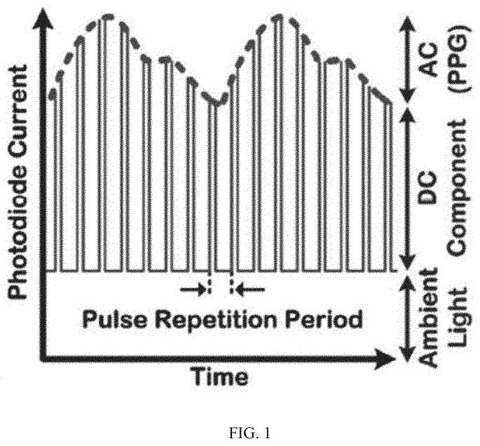

FIG. 2 is a schematic diagram to show the basic architecture of a heart rate and blood oxygen monitoring device.

FIG. 3 is a timing diagram of the ambient light signal calibration.

FIG. 4 is a flow chart of measurement data processing.

FIG. 5 is a flow chart of physiological data analysis.

### DESCRIPTION OF THE PREFERRED EMBODIMENTS

Below embodiments accompanied with drawings are used to explain the spirit of this invention to have better understanding for the person in this art, not used to limit the scope of this invention, which is defined by the claims. The applicant emphasizes the element quantity and size are schematic only. Moreover, some parts might be omitted to skeletally represent this invention for conciseness.

The ambient light (i.e., the natural light or light of an illumination device) or the reflection light from skin, surrounding tissues of arteries, bones, or veins, are all absorbed by the photodiode and mixed with the reflection of the detection of a heart rate and blood oxygen monitoring device. They are noise to the measurement and must be filtered out from the measurement result. Before a measurement, the calibration must be done.

The present invention proposes a coarse calibration and a fine calibration to filter out the interference signal of the ambient light. The coarse calibration is suitable for the stable and it is very fast. The fine calibration is suitable for the rapid-changed environment light, and it takes longer time.

First, the device generates a digital signal correspond to the ambient light and then cancels out the digital signal, noted calibration signal hereinafter, from the sensed signal. The method to generate the calibration signal is to sense the ambient light without providing the detection light in a calibration mode, i.e., the sensed signal is purely from the ambient light. The one-cycle-clock base ADC used here converts an analog voltage to a n-bit signal in one cycle to enhance conversion efficiency. In one embodiment, a 4-bit one-cycle-clock base ADC is used.

Then, the heart rate and blood oxygen monitoring device drives a light emitting element to provide a detection light, noted an operation mode. The sensed signal is calibrated by the calibration signal, i.e., to remove the calibration signal from the sensed signal in the operation mode. The control module calculates the sensed signal, which is digital, and the calculation is very fast, thus improving the sensing efficiency.

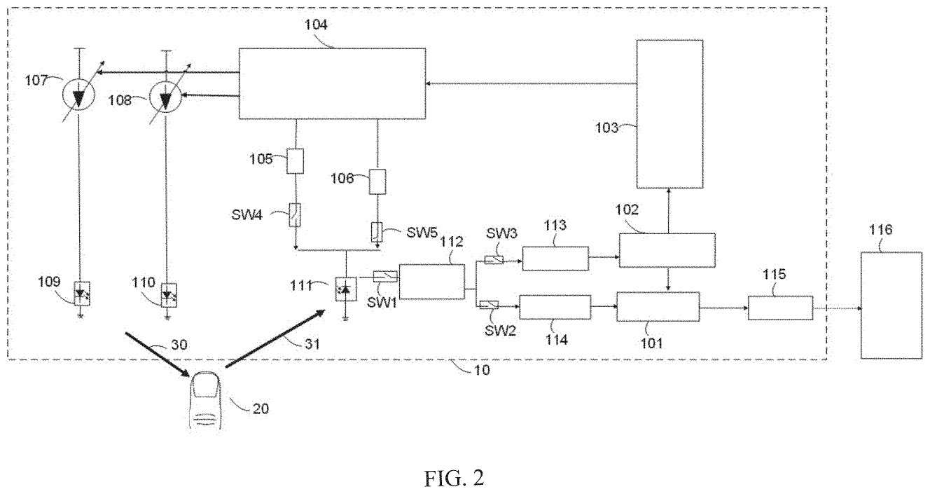

FIG. 2, a schematic diagram, shows the basic architecture of a heart rate and blood oxygen monitoring device. The heart rate and blood oxygen monitoring device 10 comprises a control module, a light emitting module, a light receiving module 111, an analog front-end module (AFE) 112, a one-cycle-clock ADC 113, an analog-to-digital converter (ADC) 114, a first ambient light calibration DAC 105, and a second ambient light calibration DAC 106.

The control module comprises a digital signal processor (DSP), a microcontroller 103, and a timing controller 104. The digital signal processor comprises a data processing unit 101 and a state machine 102. The data processing unit 101 comprises a low pass filter (not shown in the figure). The microcontroller is coupled between the state machine 102 and timing controller 104, and the state machine 102 is connected to the data processing unit 101.

The light emitting module can comprise multiple pairs of a light emitting element and its driver. In one embodiment, a red-light module (including a red-light unit 109 and its driver 107) and an infrared module are used (including an infrared-ray unit 110 and its driver 108). The drivers 107, 108 are driven by the timing controller 104, controlled by the control module. The drivers 107, 108 provide the power to turn on the light units 109, 110 to provide the detection lights. The control module controls the timing controller 104 by using a pulse signal with a pulse height (pulse intensity) and a pulse width (pulse duty), so the detection light intensity depends on the number of the emitting element.

The light receiving module 111 is implemented by a photodiode in one embodiment. The photodiode, such as a wide band silicon-base photo-diode, can receive different colors of light. The light receiving module 111 senses light, comprising a reflection light 31 of the detection light 30 from the object (Pulsatile arterial blood) 20, and convert to a current.

The analog front-end module 112, comprising an integrator and a signal amplifier (not shown in the figure), is connected to the light receiving module 111 through the first switch unit SW1 and configured to convert the sensed current to an analog signal.

The one-cycle-clock ADC 113 is connected to the state machine 102 and connected to the AFE 112 through the third switch unit SW3. The one-cycle-clock ADC 113 converts the analog signal to a calibration digital signal and transmits it to the state machine 102 in the calibration mode. The ADC 114 is connected to the data processing unit 101 and connected to the AFE 112 through the second switch SW2. The ADC 114 converts the sensed digital signal to a sensed digital signal and transmits it to the data processing unit 101 in operation mode. The SW2 or SW3 switches the device in the operation mode or the calibration mode.

In one embodiment, a first calibration DAC 105 and a second calibration DAC 106 are respectively employed to perform a coarse calibration and a fine calibration. Both DACs 105, 106 are connected to the timing controller 104 through the fourth switch unit (SW4) and the fifth switch unit (SW5) respectively. The SW4 and the SW5 is used to turn on/off the coarse and fine calibration respectively.

The switch units SW1, SW2, SW3, SW4, and SW5 are controlled by the timing controller 104 which decide to open or close circuit for operation or calibration mode.

When the SW1, the SW3 and the SW4 and/or the SW5 are closed, the SW2 is open, and the light emitting modules 109, 110 are turned off, the sensed signal is purely from the ambient light, and the heart rate and blood oxygen monitoring device enters a calibration mode. In particular, in the calibration mode, the SW4 and SW5 is used to enable the coarse correction loop and the fine correction loop respectively. Option to enable coarse correction loop or the fine correction loop. Or first enable the coarse correction loop, and then enable the fine correction loop.

In the calibration mode, the state machine 102 converts the calibration digital signal to a calibration parameter and stores it. The microcontroller 103 uses the calibration parameter to generate a control signal to the timing controller 104 to drive the DACs 105, 106 to generate calibration analog signals. At the meanwhile, the microcontroller 103 switches the SW4, and SW5 to transport the calibration analog signals to the light receiving module 111 for cancelling out the ambient light signal. One of the coarse calibration or the fine calibration is turned on, or the coarse calibration is turned on first and then switched to fine calibration circuit. At a time, only one of them is turned on.

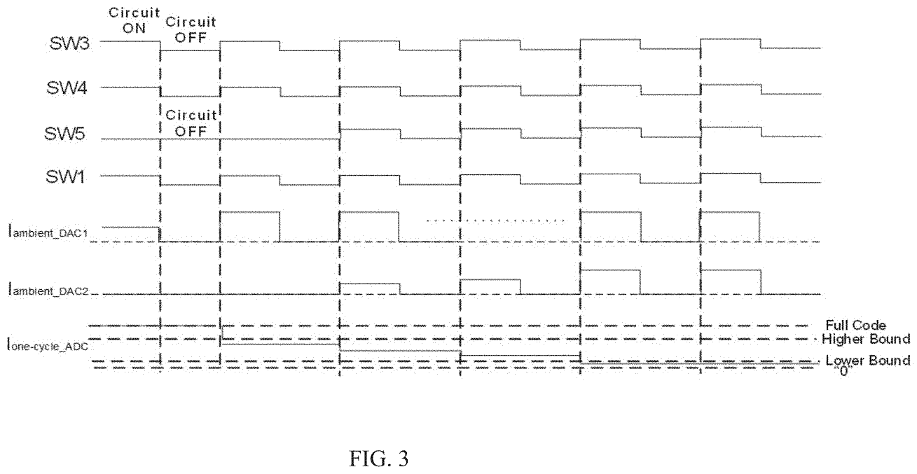

In one embodiment shown as FIG. 3, the SW4 switches on/off first and then SW5, i.e., to perform a coarse calibration first and then a fine calibration. First in the coarse calibration, the first ambient light calibration DAC 105 generates a first ambient light calibration analog signal (I_{ambient_DAC1}) first, and then in a fine calibration, the second ambient light calibration DAC 106 generates a second ambient light calibration analog signal (I_{ambient_DAC2}) to the light receiving module 111. The correct the digital signal (one-cycle clock ADC) from the upper limit (higher bound) is gradually reduced to below the lower bound (lower bound) to eliminate most of the ambient light noise. The ambient light noise is gradually cancelled out or reduced to the minimum.

The coarse calibration cancels out the ambient light interference at maximization, but the fine calibration fine checks the filtering result. If the filtering target is not reached, the heart rate and blood oxygen monitoring device will enter the fine calibration circuit again, and gradually filter the ambient light to reach the filtering target.

After the calibration is completed, the heart rate and blood oxygen monitoring device enters the operation mode when the SW3 is open, the SW1, the SW2, the SW4 and/or the SW5 are closed, and the light emitting module is turned on.

In the operation mode, the timing controller 104 drives the ambient light calibration DAC to generate the ambient light calibration analog signals for the light receiving module 111 based on the calibration parameter. The sensed signal is calibrated by the ambient light calibration analog signals, and then the ADC 114 converter the analog signal to digital signals. Then the data processing unit 101 converts the digital signals to a photoplethysmography and analyzes the heart rate and the blood oxygen.

The data processing unit 101 comprises a decimation filter, a finite impulse response filter, and a DAC mapping table correct circuit. The DAC mapping table correct circuit comprises a DAC mapping table and a correct circuit.

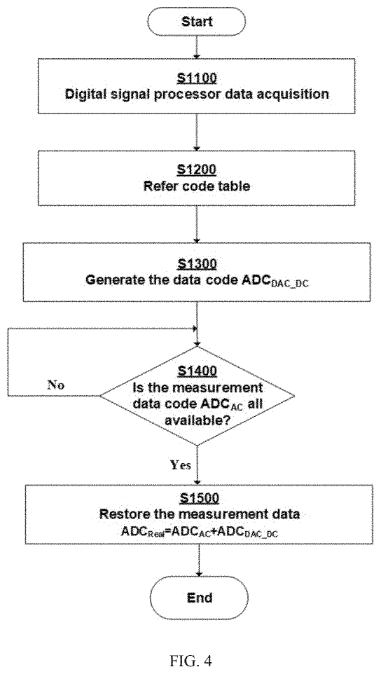

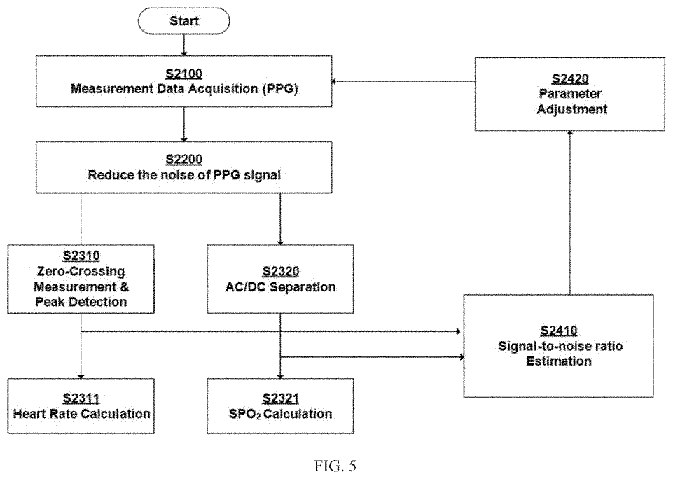

The processing steps of measuring heart rate and blood oxygen processes the measurement data first and analyzes the physiological data then, shown as FIG. 4 and FIG. 5 respectively. The former is to correct the measurement data and the latter to retrieve the heart rate and the blood oxygen saturation from the data.

FIG. 4 is a flow chart of processing the measurement data as follows.

In step S1100, the DSP acquires the sensed analog data from the ADC 114 and the calibration signal parameter.

In step S1200, the DSP retrieves a conversion parameter, noted data code or digital code (DC) here, from a mapping table. The mapping table stores the conversion parameters from an analog signal to a digital code.

In step S1300, the DSP converts the sensed analog data to a digital signal ADC_{DAC_DC} according to the conversion parameter (DC). The mapping method is very efficient.

In step S1400, the DSP waits the converts the sensed analog signal to a digital signal, noted as ADC_{AC}. The ADC_{AC} has been processed by noise filtration and period average to reduce the interference according to the calibration signal parameter, such as ambient light interference.

In step S1500, the DSP approaches the correct measurement data to achieve enough dynamic range (for example, 120 dB) by using the ADC_{DAC_DC} and ADC_{AC} according to the following formula.

ADC_{Real}=ADC_{AC}+ADC_{DAC_DC}

The method can improve the measurement, especially the high dynamic range measurement due to different human's effects including different skins, surrounding tissues of arteries, bones or veins, and so on. In one embodiment, 12-bit ADC and 12-bit DAC are used in the conversion. In one embodiment, the time controller 104 using the time-division multiplexing way to perform the red-light measurement and the infrared-ray measurement at the same time, and the data processing unit 101 can calculates the red-light measurement and the infrared-ray measurement according to the formula, i.e.,

ADC_{Red_Real}=ADC_{RED_AC}+ADC_{Red_DAC_DC}

ADC_{IR_Real}=ADC_{IR_AC}+ADC_{IR_DAC_DC}

FIG. 5 is a flow chart of analyzing the physiological data as follows.

In step S2100, the heart rate and blood oxygen monitoring device composes the photoplethysmography (PPG) based on the measurement data. The data processing unit 101 enlarge the measurement data with a gain to get the PPGs for the red-light measurement and the infrared-ray measurement. In one embodiment, the system dynamic range is 120 dB or above.

In step S2200, the heart rate and blood oxygen monitoring device reduces the noise of the PPG to increase the signal to noise ratio (SNR). In one embodiment, the decimation filter and the finite impulse response filter are used.

In step S2310, the heart rate and blood oxygen monitoring device perform a zero-crossing measurement and the peak detection to obtain the heart rate change, and then plot a heart rhythm chart by calculating the frequency of crossing the average middle line and the maximum/minimum data (peak point/valley point).

In step S2320, the heart rate and blood oxygen monitoring device separates the DC component and the AC component from the PPG. The AC component has a signal variation. In one embodiment, the heart rate and blood oxygen monitoring device respectively obtains the AC component (AC_{R}) and the DC component (DC_{R}) from the red-light measurement, and the AC component (AC_{IR}) and the DC component (DC_{IR}) from the infrared-ray measurement. In step S231, the heart rate and blood oxygen monitoring device calculates the blood oxygen saturation (SPO₂) based on the DC component and the AC component by following formula.

$${SPO}_{2} = 110 - 25 R \cdot R = \frac{\frac{{AC}_{R}}{{DC}_{R}}}{\frac{{AC}_{IR}}{{DC}_{IR}}}$$

In step S2410, the heart rate and blood oxygen monitoring device estimates the SNR to decide to optimize further or not. If yes, the device generates new adjustment parameter in step S2420 and perform the step S2100 again to generate a better PPG.

The heart rate and blood oxygen monitoring device can be further equipped with a data transmission module 115 according the substantial requirements, the transmission module 115 could comprise a first input first output (FIFO) and different communication/protocol interfaces.

In one embodiment, the heart rate and blood oxygen monitoring device comprises a data transmission module 115 and a Bluetooth chip module 116. The data transmission module 115 can transmit the measurement data, the DC component and the AC component of the PPG, and the SPO₂ to an external device via the Bluetooth chip module 116. The measurement data, the DC component and the AC component, and the SPO₂ are stored in the state machine 102.

The heart rate and blood oxygen monitoring device uses one-cycle-clock ADC and the ambient light calibration DAC to quickly calibrate the ambient-light interference. The present invention can improve the accuracy and the performance of monitoring heart rate and blood oxygen without affecting user's operating. Moreover, the heart rate and blood oxygen monitoring device can be implemented by a circuit-integrated chip to reach the goal of miniaturization.

The heart rate and blood oxygen monitoring device can be an independent heart rate and blood oxygen measurement equipment or can be integrated on a wearable device.

What is claimed is:

1. A heart rate and blood oxygen monitoring device, comprising:
a red-light emitting element driven by a red-light driver to emit a red-light detection light, and an infrared-ray emitting element driven by an infrared-ray driver to emit an infrared-ray detection light;
a light receiving module connected to an analog front-end module through a first switch unit to convert the detection lights to an analog signal;
an analog-to-digital converter connected to the analog front-end module through a second switch unit to convert the analog signal to an operation digital signal;
a one-cycle-clock ADC connected to the analog front-end module through a third switch unit to convert the analog signal to a calibration digital signal;
a digital signal processor connected to the analog-to-digital converter and the one-cycle-clock ADC to convert the calibration digital signal and the operation digital signal to a calibration parameter and a measurement data respectively;
a first ambient light calibration DAC connected to the light receiving module through a fourth switch unit to transmit an ambient light calibration analog signal to the light receiving module;
a timing controller configured to drive the red-light driver, the infrared-light driver, and the first ambient light calibration DAC; and
a microcontroller connected to the digital signal processor and the timing controller, the microcontroller controls the heart rate and blood oxygen monitoring device into an operation mode or a calibration mode by switching the first switch unit, the second switch unit, the third switch unit, and the fourth switch unit, wherein the red-light detection light and the infrared-ray detection light can be partly absorbed and partly reflected to measure a heart rate and a blood oxygen saturation value.

2. The heart rate and blood oxygen monitoring device according to claim 1, wherein the operation mode is defined when the first switching unit is switched to close, the second switching unit is switched to close, the third switching unit is switched to open, and the fourth switching unit is switched to close, the red-light driver drives the red-light emitting element, and the infrared-ray driver drives the infrared-ray emitting element.

3. The heart rate and blood oxygen monitoring device according to claim 1, wherein the calibration mode is defined when the first switch unit is switched to close, the second switch unit is switched to open, the third switch unit is switched to close, and the fourth switch unit is switched to close, the red-light driver does not drive the red-light emitting element, and the infrared-ray driver does not drive the infrared-ray emitting element.

4. The heart rate and blood oxygen monitoring device according to claim 1, further comprising a second ambient light calibration DAC, wherein the first ambient light calibration DAC and the second ambient light calibration DAC are configured to provide a coarse ambient light calibration analog signal and a fine ambient light calibration analog signal respectively.

5. The heart rate and blood oxygen monitoring device according to claim 4, wherein only one of the first ambient light calibration DAC and the second ambient light calibration DAC is turned on during a time.

6. The heart rate and blood oxygen monitoring device according to claim 1, wherein the digital signal processor comprises:
a state machine is configured to receive and store the calibration parameter; and
a data processing unit is configured to receive the measurement data and analyze the heart rate and the blood oxygen saturation value.

7. The heart rate and blood oxygen monitoring device according to claim 6, wherein the data processing unit comprises a decimation filter, a finite impulse response filter, a DAC mapping table, and a correct circuit, wherein the decimation filter and the finite impulse response filter are configured to reduce the noise of the measurement data, the DAC map table is configured to store an analog-to-digital convert correction data, and the correct circuit is configured to correct the measurement data according to the analog-to-digital convert correction data.

8. The heart rate and blood oxygen monitoring device according to claim 1, further comprising a data transmission module and a Bluetooth module configured to transmit the measurement data, a DC component and an AC component of the PPG, and the blood oxygen saturation value to an external electronic device.

9. The heart rate and blood oxygen monitoring device according to claim 1, wherein the heart rate and blood oxygen monitoring device is a medical heart rate and blood oxygen monitoring device or can be integrated into a medical measuring device.

10. The heart rate and blood oxygen monitoring device according to claim 1, wherein the heart rate and blood oxygen monitoring device can be integrated into a wearable device.
